(19) United States
(12) Patent Application Publication
(10) Pub. No.: US 2025/0213387 A1
(43) Pub. Date: Jul. 3, 2025

(54) INCLINED APPLICATOR TIPS FOR COOLING DEVICES

(71) Applicant: RECENSMEDICAL, INC., Hwaseong-si, Gyeonggi-do (KR)

(72) Inventor: Gun-Ho KIM, Hwaseong-si, Gyeonggi-do (KR)

(21) Appl. No.: 18/850,995

(22) PCT Filed: Mar. 30, 2023
(86) PCT No.: PCT/IB2023/000173

§ 371 Date: Sep. 25, 2024

Related U.S. Application Data
(60) Provisional application No. 63/326,094, filed on Mar. 31, 2022.

(51) Int. Cl. A61F 7/00

(52) U.S. Cl. CPC A61F 7/00; A61F 2007/0063; A61F 2007/0087

(57) ABSTRACT

Proposed is an inclined applicator tip adapted to be operably coupled to a handheld cooling device. The inclined applicator tip may include an opened portion, at least a portion of an edge of the opened portion comprising a c-shape opening. The inclined applicator tip may also include an elongated body portion coupled to the opened portion, the longest dimension of the elongated body portion forming an angle of inclination angle of inclination (θ) with respect to the plane which is parallel to the c-shape opening, the elongated body portion being substantially parallel to the cooling axis of the handheld cooling device when operably coupled. The inclined applicator tip may further include a mounting portion adapted to be operably coupled to the cooling device, the mounting portion and the opened portion being coupled to the elongated body portion at opposite ends of the longest dimension of the elongated body portion.

### CROSS-REFERENCE

This application claims the benefit of U.S. Provisional Patent Application No. 63/326,094 filed on Mar. 31, 2022, which is incorporated by reference in its entirety.

### BACKGROUND

Cooling devices are used in many analgesic, anesthetic and cryosurgical applications. Accordingly, it is desirable that applicator tips for a cooling device allow access to the subject area during use of a cooling device to improve therapeutic procedures.

### SUMMARY

In one aspect, described herein are inclined applicator tips adapted to be operably coupled to a handheld cooling device. In some aspects, the inclined applicator tip comprises an opened portion wherein at least a portion of an edge of the opened portion comprises a c-shape opening. In some aspects, the inclined applicator tip comprises an elongated body portion coupled to the opened portion, wherein the longest dimension of the elongated body portion forms an angle of inclination (θ) with respect to the plane which is parallel to the c-shape opening, wherein the elongated body portion is substantially parallel to the cooling axis of the handheld cooling device when operably coupled; and a mounting portion adapted to be operably coupled to the cooling device; wherein the mounting portion and the opened portion are coupled to the elongated body portion at opposite ends of the longest dimension of the elongated body portion.

In some aspects, the inclined applicator tip comprises an opened portion which comprises a circular, an elliptical, or a polygonal shape. In some aspects, the inclined applicator tip comprises an opened portion which comprises a circular shape wherein the portion of the circular shape which forms the opening comprises a 90° arc. In some aspects, the opened portion comprises a circular shape and the portion of the circular shape which forms the opening comprises a 180° arc. In some aspects, the opened portion comprises a circular shape and the portion of the circular shape which forms the opening comprises a 270° arc.

In some aspects, the angle of inclination (θ) is about 60°. In some aspects, the angle of inclination (θ) is about 70°. In some aspects, wherein the angle of inclination (θ) is about 80°. In some aspects, the angle of inclination (θ) is about 90°. In some aspects, the opened portion comprises an inner radius of at least 5 mm, 10 mm, 15 mm, 20 mm, 30 mm, or 50 mm. In some aspects, the longest dimension of the elongated body portion is no more than about 250 mm. In some aspects, the longest dimension of the elongated body portion is at least about 15 mm. In some aspects, the handheld cooling device is a cryo-genic cooling device. In some aspects, the angle of inclination (θ) is about 60°. In some aspects, the angle of inclination (θ) is about 70°. In some aspects, the angle of inclination (θ) is about 80°. In some aspects, the angle of inclination (θ) is about 90°.

In some aspects, the opened portion comprises a circular shape and the portion of the circular shape which forms the opening with a 90° arc. In some aspects, the opened portion comprises a circular shape and the portion of the circular shape which forms the opening with a 180° arc. In some aspects, the angle of inclination (θ) is about 60° and the opened portion comprises an inner radius of at least 5 mm.

In some aspects, the mounting portion comprises a magnetic coupling element adapted to be operably coupled to the cooling device. In some aspects, the magnetic coupling element is one or more permanent magnets. In some aspects, the magnetic coupling element is made of a ferromagnetic material, and the cooling device comprises one or more magnets configured to attract the ferromagnetic material when the inclined applicator tip is coupled to the cooling device.

In another aspect described herein are methods of cooling a target surface using a handheld cooling device. In some aspects, the method includes providing an inclined applicator tip that is adapted to be operably coupled to the handheld cooling device, wherein the inclined applicator tip comprises an opened portion, an elongated body portion coupled to the opened portion, and a mounting portion adapted to be operably coupled to the handheld cooling device.

In some aspects, the method includes selecting an orientation of the opened portion with respect to a cooling axis of the handheld cooling device, wherein the orientation of the opened portion forms an angle of inclination (θ) with respect to the cooling axis; and cooling the target surface by delivering the cooling medium to the target surface along the cooling axis.

In some aspects, selection is performed by mounting the applicator tip to the handheld cooling device in a position selected from three discreet rotational orientations. In some aspects, the selection is performed by selecting an orientation from a continuum of rotational orientations using a rotatable mount of the handheld cooling device. In some aspects, the cooling medium is a cryogen. In some aspects, the angle of inclination and the user selected orientation are designed to minimize a reduction of cryogen flow into the cooling device caused by gravity. In some aspects, the method further comprises applying a treatment to the target surface concurrently or near simultaneously with the cooling. In some aspects, the treatment comprises an injection, a laser treatment, a surgery, or a drug application. In some aspects, the treatment is performed through the opened portion of the inclined applicator tip.

Additional aspects and advantages of the present disclosure will become readily apparent to those skilled in this art from the following detailed description, wherein only illustrative aspects of the present disclosure are shown and described. As will be realized, the present disclosure is capable of other and different aspects, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative aspects, in which the principles of the invention are utilized, and the accompanying drawings (also “Figure” and “FIG.” herein), of which:

FIG. 1 illustrates an exemplary non-inclined applicator tip installed on an exemplary cryogen-based cooling device. The top panel illustrates angle of inclination for a cryogenic cooling device when using a non-inclined tip for a vertical target tissue, where cryogen flow is reduced by gravity. The lower panel illustrates an optimal inclement angle for a cryogenic cooling device when using a non-inclined tip for a horizontal target surface, where cryogen flow is not substantially reduced by gravity.

FIG. 2 illustrates an exemplary inclined applicator tip installed on an exemplary cryogen-based cooling device. The top and bottom panels illustrate that an inclined applicator tip can allow optimal cryogen flow when an exemplary cryogen-based therapeutic device is applied to a target tissue at any angle.

FIG. 3 illustrates exemplary advantages of an inclined applicator tip in access to an exemplary therapeutic application site. (A) illustrates that use of a fully-enclosed, non-inclined applicator tip obstructs access to the target therapeutic site. (B) and (C) illustrate that use of an opened shaped, inclined applicator tip allows access to the target therapeutic site for simultaneous or near-simultaneous administration of an injection (B) and/or laser therapy (C).

FIG. 4 illustrates exemplary measurements of an angle of inclination of an inclined applicator tip with respect to a target tissue.

FIG. 5 illustrates a variety of example “opened” shapes that can be used in an inclined opened applicator tip, which can comprise (A) elliptical, (B) circular, (C) triangular, (D) pentagonal, (E) hexagonal, (F) octagonal, or (G) decagonal.

FIG. 6 illustrates a perspective view of an example opened applicator tip.

FIG. 7 illustrates (A) a top view and (B) a bottom view of an example opened applicator tip.

FIG. 8 illustrates (A) a left side view, (B) a right side view, (C) a rear view, and (D) a front view of an example opened applicator tip.

### DETAILED DESCRIPTION

While various aspects of the invention have been shown and described herein, it will be obvious to those skilled in the art that such aspects are provided by way of example only. Numerous variations, changes, and substitutions can occur to those skilled in the art without departing from the invention. It should be understood that various alternatives to the aspects of the invention described herein can be employed.

### Devices

In one aspect, described herein are devices for use with a handheld cooling device.

In one aspect, described herein are inclined applicator tips adapted to be operably coupled to a cooling device. In some instances, the cooling device is a handheld cooling device. In some instances, the cooling device is a therapeutic cooling device. In some instances, the handheld cooling device is a therapeutic cooling device. In some aspects, the inclined applicator tips comprise an opened portion wherein at least a portion of an edge of the opened portion comprises a c-shape opening. In some aspects, the inclined applicator tips comprise an elongated body portion coupled to the opened portion, wherein the longest dimension of the elongated body portion forms an angle of inclination (θ) with respect to the plane which is parallel to the c-shape opening. In some aspects, the elongated body portion is substantially parallel to the cooling axis of the handheld cooling device when operably coupled, and a mounting portion adapted to be operably coupled to the cooling device. In some aspects, the mounting portion and the opened portion are coupled to the elongated body portion at opposite ends of the longest dimension of the elongated body portion.

In some aspects, the inclined applicator tip comprises an opened portion which comprises a circular, an elliptical, or a polygonal shape. In some aspects, the inclined applicator tip comprises an opened portion which comprises a circular shape wherein the portion of the circular shape which forms the opening comprises a 90° arc. In some aspects, the opened portion comprises a circular shape and the portion of the circular shape which forms the opening comprises an 180° arc. In some aspects, the opened portion comprises a circular shape and the portion of the circular shape which forms the opening comprises a 270° arc.

In some aspects, the angle of inclination (θ) is about 60°. In some aspects, the angle of inclination (θ) is about 65°. In some aspects, the angle of inclination (θ) is about 70°. In some aspects, the angle of inclination (θ) is about 75°. In some aspects, wherein the angle of inclination (θ) is about 80°. In some aspects, the angle of inclination (θ) is about 85°. In some aspects, the angle of inclination (θ) is about 90°. In some embodiments, the opened portion comprises an inner radius of at least 5 mm, 10 mm, 15 mm, 20 mm, 25 mm, 30 mm, 35 mm, 40 mm, 45 mm, or 50 mm.

In some aspects, the longest dimension of the elongated body portion is no more than about 150 mm, about 200 mm, about 250 mm, about 300 mm. In some aspects, the longest dimension of the elongated body portion is at least about 15 mm, at least about 20 mm, at least about 25 mm, at least about 30 mm, at least about 35 mm, at least about 40 mm, at least about 45 mm, at least about 50 mm. In some aspects, the handheld cooling device is a cryogenic cooling device.

In some aspects, the opened portion comprises a circular shape and the portion of the circular shape which forms the opening with a 90° arc. In some aspects, the opened portion comprises a circular shape and the portion of the circular shape which forms the opening with a 180° arc. In some aspects, the angle of inclination (θ) is about 60° and the opened portion comprises an inner radius of at least 5 mm.

In some aspects, the mounting portion comprises a magnetic coupling element adapted to be operably coupled to the cooling device. In some aspects, the magnetic coupling element is one or more permanent magnets. In some aspects, the magnetic coupling elements are made of a ferromagnetic material, and the cooling device comprises one or more magnets configured to attract the ferromagnetic material when the inclined applicator tip is coupled to the device.

### Use of Inclined Applicator Tip

Also described herein are methods for use of devices described herein to provide cooling to a subject.

In some aspect described herein are methods of cooling a target surface using a handheld cooling device. In some aspects, the handheld device comprises, or coupled to, an inclined applicator tip that is adapted to be operably coupled to the handheld cooling device. In some instances, the inclined applicator tip comprises an opened portion, an elongated body portion coupled to the opened portion, and a mounting portion adapted to be operably coupled to the handheld cooling device.

In some aspects, an orientation of the opened portion can be selected with respect to a cooling axis of the handheld cooling device, such that the orientation of the opened portion forms an angle of inclination (θ) with respect to the cooling axis. Once the orientation is selected, the target surface can be cooled by delivering the cooling medium from the handheld cooling device to the target surface. In some instances, the cooling medium can be delivered to the target surface substantially along the cooling axis. In some instances, the cooling medium can be delivered to the target surface by spreading along the cooling axis, for example by diffusion of a cooling jet.

In some aspects, selection of the orientation of the opened portion is performed by mounting the applicator tip to the handheld cooling device in a position selected from three discreet rotational orientations. In some aspects, the selection of the orientation of the opened portion is performed by selecting an orientation from a continuum of rotational orientations using a rotatable mount of the handheld cooling device. For example, a device comprising an inclined applicator tip with a fixed angle of inclination and a rotatable mount can accommodate target tissues which are tilted to any orientation, such as tilted up or tilted down with opposite magnitudes. In this example, the orientation of the device can be maintained with respect to gravity by rotating the tip 180°.

In some aspects, the cooling medium is a cryogen. In some instances, a cryogen is a gas cryogen, liquid cryogen, or non-liquid cryogen (e.g., solid cryogen). In some instances, a cryogen is any appropriate substance that can cool down the target surface to −5° C., −4° C., −3° C., −2° C., −1° C., 0° C., 1° C., 2° C., 3° C., 4° C., or 5° C.

In some aspects, the angle of inclination and the orientation are selected to minimize a reduction of cryogen flow or maximize the cryogen flow into the cooling device caused by gravity. In some instances, the angle of inclination and the orientation are selected to optimize discharge pressure of the cryogen in the handheld cooling device. In some instances, the angle of inclination and the orientation are selected to optimize discharged amount of the cryogen to the target surface per application.

In some aspects, a treatment to the target surface can be concurrently or near simultaneously (e.g., within 1 second, 2 seconds, 3seconds, 4 seconds, 5 seconds, 10 seconds after cooling the target surface or before cooling the target surface) applied with the cooling of the target surface. In some aspects, the treatment is performed through the opened portion of the inclined applicator tip. In some aspects, the treatment comprises an injection, a laser treatment, a surgery, or a drug application. For example, a cooling treatment can be combined with an injection in order to provide a vaccination to a subject with minimal pain or swelling at the injection site. A single technician or a team of two or more technicians can administer the vaccine by first applying the cooling device, either by placing the applicator tip onto a target surface of the subject. The technician or a second technician can then inject either during cooling by the cooling device or by injecting within a few second of the conclusion of a cooling cycle.

### Terms and Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The terminology used herein is for the purpose of describing particular cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”.

As used herein, the term “comprise” or variations thereof such as “comprises” or “comprising” are to be read to indicate the inclusion of any recited feature but not the exclusion of any other features. Thus, as used herein, the term “comprising” is inclusive and does not exclude additional, unrecited features. In some aspects of any of the compositions and methods provided herein, “comprising” can be replaced with “consisting essentially of” or “consisting of.” The phrase “consisting essentially of” is used herein to require the specified feature(s) as well as those which do not materially affect the character or function of the claimed disclosure. As used herein, the term “consisting” is used to indicate the presence of the recited feature alone. As used herein, the term “and/or” includes any and all combinations of one or more of the associated listed items.

In the drawings, the thicknesses of layers and regions are exaggerated for clarity, and elements or layers are referred to as “on” or “on” the other elements or layers includes not only cases where they are directly above the other components or layers, but also cases where there are other layers or other components interposed in the middle. The same reference numerals throughout these specifications refer to the same elements unless indicated otherwise. In addition, components that have the same functions within the scope of the same idea shown in the drawings of each embodiment are described using the same reference numerals, and redundant descriptions thereof are omitted.

The numbers (e.g., first, second, etc.) used in the description of the present disclosure are merely identification symbols for distinguishing one component from other components.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

Unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously. Thus, the term “thermally coupled” refers any relationship between two materials or objects where the heat is transferred by direct or indirect contact (e.g., via vapor, air, liquid, mechanical forces, etc.).

In some aspects, the term ‘target’ refers to an object that is cooled by using a cooling system. For example, in some aspects, the target is a target intended to be put into an anesthetic state or a painless state in order to receive a medical procedure. Specifically, the target comprises a part of a body including nerves, such as eyes, skin, or gum with disease. In another example, the target comprises a target for skin cosmetic treatment using cooling. Specifically, the target can comprise a part of a body including a point that can be removed by cooling a local area, warts, callus, etc., or a part of a body requiring local anesthesia during laser treatment such as hair removal, peeling, and Botox treatment. In some aspects, the target can include cells or tissues for scientific experiments (e.g., cell culture, tissue culture, cultured cells, cultured tissue). Alternatively, and/or additionally, the target can include any containers (e.g., glass vial, glass substrate, petri dish, etc.) containing cells or tissues.

As used herein, the term ‘cooling’ refers to reducing the temperature of a target to be cooled. In some aspects, the cooling is accomplished by applying cooling energy to the target to be cooled and absorbing the heat energy of the target to be cooled. In some aspects, the cooling energy can refer to the escaping of heat by cooling and can be understood as a concept for expressing the decrease of thermal energy. For example, cooling can be conducted by applying cooling energy through a cooling medium, which in some aspects can further cool a contact tip, or by applying cooling energy to the tip, and ‘contacting’ the cooling medium and/or a cooled tip to a target to be cooled so that the cooling energy can be transmitted or applied to the target.

In another example, cooling can be conducted by ‘injecting’ or jetting a coolant, air, gas, or a liquid cryogen to apply cooling energy to the target to be cooled. Injecting or jetting can include producing a controlled aerosol spray of liquid cryogen directly or indirectly on to a target surface to be cooled. In other words, it should be understood as a comprehensive concept including various methods of applying cooling energy to an object to be cooled. Hereinafter, for convenience of description, cooling a target through a non-contact cooling device which operates by applying a cryogen spray to the target is described as a main embodiment, but the technical idea of the present specifications is not limited thereto.

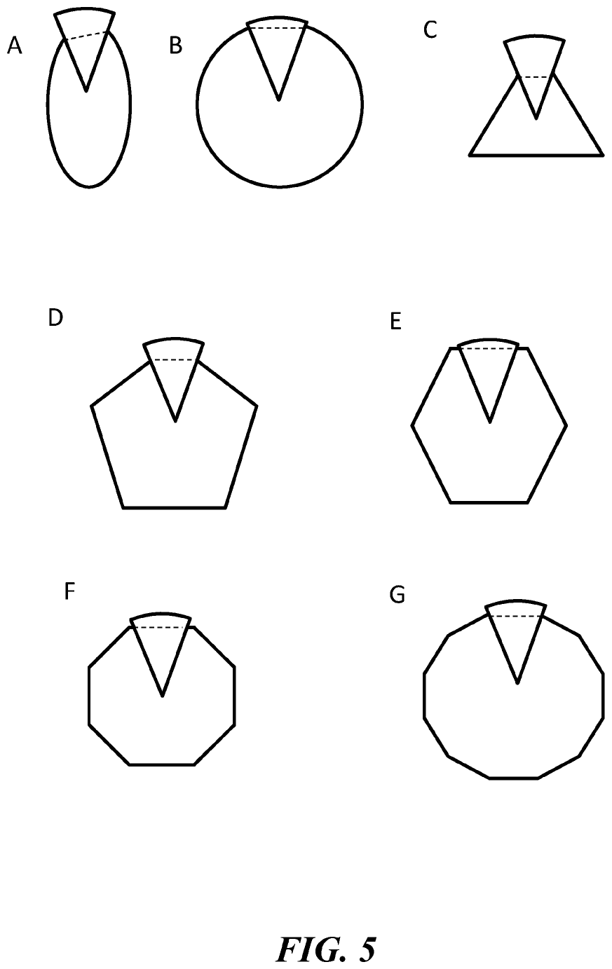

As used herein, “opened” generally refers to an object or feature which has a cross sectional shape wherein at least a portion of an edge of the shape has been removed along an arc to form an opening, such as but not limited to examples illustrated in FIG. 5. The cross-sectional shape can for example be circular, elliptical, triangular, rectangular, pentagonal, hexagonal, heptagonal, octagonal, nonagonal, decagonal, or any other regular or irregular polygon shape such that the resulting shape with the arc removed to form an access opening which in some aspects can resembles a capital letter “C”.

As used herein, “cooling axis” generally refers to the line along which a cooling device operates. For example, in a cryogen-based cooling device, the cooling axis is the direction of the cryogen spray. Examples of cryogen-based cooling devices include devices which apply a spray or aerosol of a liquified cryogen-gas such as but not limited to liquid He, Ar, N₂, O₂, CO₂, H₂, methane, ethane, propane, or combinations or mixtures thereof to reduce the temperature of surfaces that the spray or aerosol comes into contact with.

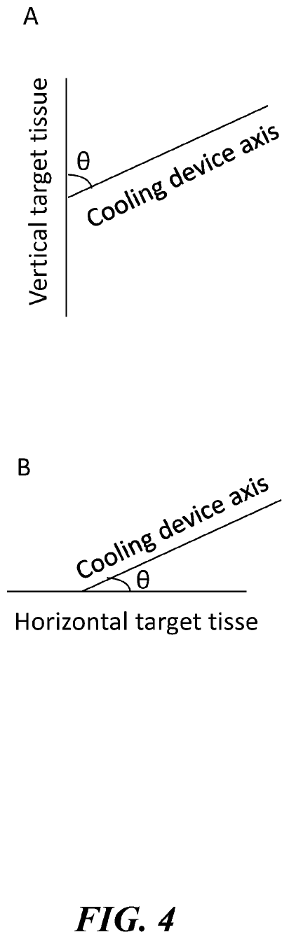

As used herein, “angle of inclination (θ)” is measured as the angle between the longitudinal cooling axis of a cooling device and the surface of a target tissue when the cooling device is operably coupled to an inclined applicator tip, for example the angle described in FIG. 4. An inclined applicator tip can set the angle of inclination by placing the contact surface of the applicator tip at the angle of inclination with respect to the longitudinal cooling axis of the therapeutic device.

As used herein, “non-contact” refers to a cooling device which does not rely on direct induction of energy between a heat exchanger and the target site. Examples of non-contact cooling devices include cryogen spray-based cooling devices. Importantly, non-contact does not indicate that no part of the device is in contact with the subject or target site. Non-contact cooling devices can include guide tips, such as those described herein, which have a contact surface and can be used to fix the distance between a non-contact cooling element, such as a cryogen spray nozzle, and a target site.

As used herein, “simultaneous” refers to events that occur at substantially the same time, wherein two events that within a second of each other are simultaneous.

As used herein, “near-simultaneous” refers to an event occurring at a time that is no more than about 60 seconds after an application of a cooling device as measured from the completion of a full cooling cycle. For example, an event occurring about 1 second to about 60 seconds after the completion of the cooling cycle would be “near-simultaneous”.

### Inclined Applicator Tip

In one aspect, described herein are inclined applicator tips for use with a cooling device. In some aspects, the cooling device is a handheld cooling device. In some aspects, the cooling device is a cooling device. In some aspects, the cooling device is an electrothermal cooling device. In some aspects, the cooling device is a cryogenic cooling device. In some aspects, the cooling device is a non-contact cooling device. In some aspects, the cooling device is a contact-based cooling device. In some aspects, the cooling device is any suitable device that can be used with the inclined applicator tip, for example, as described in U.S. Patent Application Pub. Nos. US20190175394A1, US20200309436A1, US20200054483A1, US20210290430A1,US20190167330A1, International Patent Application Pub. Nos. WO2018221848A1,WO2022015066A1, WO2019107607A1, WO2021246580A1, WO2022015067A1,WO2020022858A1, Korean Patent Application Nos. KR20190114710A, KR20190124971A, KR20210101187A, KR20190063724A, KR20190063723A, KR20210030315A, KR20200070200A, KR20190124974A, KR20200012677A, KR20190063722A, KR20190124970A, KR20200124635A, KR20190124973A, KR20190124969A and U.S. Pat Nos. 11,207,488, 11,154,417, 11,241,332, which are incorporated by references herein.

In some instances, the inclined applicator tip can comprise an opened portion 601 which comprises a contact surface 701 at the lower end of the applicator tip, which can be applied to the target.

In some aspects, the opened portion 601 has an inner radius 602 and an outer radius 604. In some aspects, the inner and outer radii 602 and 604 can be described independently as a radial distance from the cooling axis of the cooling device. The inner and outer radii 602 and 604 can also be described by a distance of either of the radii from the cooling axis and a thickness of the opened portion of the applicator tip.

In some aspects, the inner radius or the outer radius of the opened portion is independently about 2 mm to about 100 mm. In some aspects, the inner radius or the outer radius of the opened portion is independently about 2 mm to about 3 mm, about 2 mm to about 4 mm, about 2 mm to about 5 mm, about 2 mm to about 7 mm, about 2 mm to about 10 mm, about 2 mm to about 20 mm, about 2 mm to about 30 mm, about 2 mm to about 40 mm, about 2 mm to about 50 mm, about 2 mm to about 75 mm, about 2 mm to about 100 mm, about 3 mm to about 4 mm, about 3 mm to about 5 mm, about 3 mm to about 7 mm, about 3 mm to about 10 mm, about 3 mm to about 20 mm, about 3 mm to about 30 mm, about 3 mm to about 40 mm, about 3 mm to about 50 mm, about 3 mm to about 75 mm, about 3 mm to about 100 mm, about 4 mm to about 5 mm, about 4 mm to about 7 mm, about 4 mm to about 10 mm, about 4 mm to about 20 mm, about 4 mm to about 30 mm, about 4 mm to about 40 mm, about 4 mm to about 50 mm, about 4 mm to about 75 mm, about 4 mm to about 100 mm, about 5 mm to about 7 mm, about 5 mm to about 10 mm, about 5 mm to about 20 mm, about 5 mm to about 30 mm, about 5 mm to about 40 mm, about 5 mm to about 50 mm, about 5 mm to about 75 mm, about 5 mm to about 100 mm, about 7 mm to about 10 mm, about 7 mm to about 20 mm, about 7 mm to about 30 mm, about 7 mm to about 40 mm, about 7 mm to about 50 mm, about 7 mm to about 75 mm, about 7 mm to about 100 mm, about 10 mm to about 20 mm, about 10 mm to about 30 mm, about 10 mm to about 40 mm, about 10 mm to about 50 mm, about 10 mm to about 75 mm, about 10 mm to about 100 mm, about 20 mm to about 30 mm, about 20 mm to about 40 mm, about 20 mm to about 50 mm, about 20 mm to about 75 mm, about 20 mm to about 100 mm, about 30 mm to about 40 mm, about 30 mm to about 50 mm, about 30 mm to about 75 mm, about 30 mm to about 100 mm, about 40 mm to about 50 mm, about 40 mm to about 75 mm, about 40 mm to about 100 mm, about 50 mm to about 75 mm, about 50 mm to about 100 mm, or about 75 mm to about 100 mm. In some aspects, the inner radius or the outer radius of the opened portion is independently about 2 mm, about 3 mm, about 4 mm, about 5 mm, about 7 mm, about 10 mm, about 20 mm, about 30 mm, about 40 mm, about 50 mm, about 75 mm, or about 100 mm. In some aspects, the inner radius or the outer radius of the opened portion is independently at least about 2 mm, about 3 mm, about 4 mm, about 5 mm, about 7 mm, about 10 mm, about 20 mm, about 30 mm, about 40 mm, about 50 mm, or about 75 mm. In some aspects, the inner radius or the outer radius of the opened portion is independently at most about 3 mm, about 4 mm, about 5 mm, about 7 mm, about 10 mm, about 20 mm, about 30 mm, about 40 mm, about 50 mm, about 75 mm, or about 100 mm.

In some aspects, the opening 606 of the opened portion 601 is described by a circular arc. The opening of the opened portion can be defined by a circular arc, or by the deletion of a portion of a cross-sectional shape described by a circular arc, for example in the manner demonstrated in FIG. 5A-G, for various cross sectional contact surface shapes. In some aspects, the cross sectional shape of the contact surface 701 can be described by a clearance distance between the two points of the cross section which are furthest away from each other.

In some aspects, the clearance distance is about 2 mm to about 100 mm. In some aspects the clearance distance is about 2 mm to about 3 mm, about 2 mm to about 4 mm, about 2 mm to about 5 mm, about 2 mm to about 7 mm, about 2 mm to about 10 mm, about 2 mm to about 20 mm, about 2 mm to about 30 mm, about 2 mm to about 40 mm, about 2 mm to about 50 mm, about 2 mm to about 75 mm, about 2 mm to about 100 mm, about 3 mm to about 4 mm, about 3 mm to about 5 mm, about 3 mm to about 7 mm, about 3 mm to about 10 mm, about 3 mm to about 20 mm, about 3 mm to about 30 mm, about 3 mm to about 40 mm, about 3 mm to about 50 mm, about 3 mm to about 75 mm, about 3 mm to about 100 mm, about 4 mm to about 5 mm, about 4 mm to about 7 mm, about 4 mm to about 10 mm, about 4 mm to about 20 mm, about 4 mm to about 30 mm, about 4 mm to about 40 mm, about 4 mm to about 50 mm, about 4 mm to about 75 mm, about 4 mm to about 100 mm, about 5 mm to about 7 mm, about 5 mm to about 10 mm, about 5 mm to about 20 mm, about 5 mm to about 30 mm, about 5 mm to about 40 mm, about 5 mm to about 50 mm, about 5 mm to about 75 mm, about 5 mm to about 100 mm, about 7 mm to about 10 mm, about 7 mm to about 20 mm, about 7 mm to about 30 mm, about 7 mm to about 40 mm, about 7 mm to about 50 mm, about 7 mm to about 75 mm, about 7 mm to about 100 mm, about 10 mm to about 20 mm, about 10 mm to about 30 mm, about 10 mm to about 40 mm, about 10 mm to about 50 mm, about 10 mm to about 75 mm, about 10 mm to about 100mm, about 20 mm to about 30 mm, about 20 mm to about 40 mm, about 20 mm to about 50 mm, about 20 mm to about 75 mm, about 20 mm to about 100 mm, about 30 mm to about 40 mm, about 30 mm to about 50 mm, about 30 mm to about 75 mm, about 30 mm to about 100 mm, about 40 mm to about 50 mm, about 40 mm to about 75 mm, about 40 mm to about 100 mm, about 50 mm to about 75 mm, about 50 mm to about 100 mm, or about 75 mm to about 100 mm. In some aspects, the clearance distance is about 2 mm, about 3 mm, about 4 mm, about 5 mm, about 7 mm, about 10 mm, about 20 mm, about 30 mm, about 40 mm, about 50 mm, about 75 mm, or about 100 mm. In some aspects, the clearance distance is at least about 2 mm, about 3 mm, about 4 mm, about 5 mm, about 7 mm, about 10 mm, about 20 mm, about 30 mm, about 40 mm, about 50 mm, or about 75 mm. In some aspects, the clearance distance is at most about 3 mm, about 4 mm, about 5 mm, about 7 mm, about 10 mm, about 20 mm, about 30 mm, about 40 mm, about 50 mm, about 75 mm, or about 100 mm.

In some aspects, the opening of the opened portion is defined by a gap distance between the end points of the opening (e.g. the gap width of the opening between the points). For example, by measuring the length of the dashed lines in FIG. 5A-G or in 606.

In some aspects, the gap width is about 2 mm to about 100 mm. In some aspects the gap width is about 2 mm to about 3 mm, about 2 mm to about 4 mm, about 2 mm to about 5 mm, about 2 mm to about 7 mm, about 2 mm to about 10 mm, about 2 mm to about 20 mm, about 2 mm to about 30 mm, about 2 mm to about 40 mm, about 2 mm to about 50 mm, about 2 mm to about 75 mm, about 2 mm to about 100 mm, about 3 mm to about 4 mm, about 3 mm to about 5 mm, about 3 mm to about 7 mm, about 3 mm to about 10 mm, about 3 mm to about 20 mm, about 3 mm to about 30 mm, about 3 mm to about 40 mm, about 3 mm to about 50 mm, about 3 mm to about 75 mm, about 3 mm to about 100 mm, about 4 mm to about 5 mm, about 4 mm to about 7 mm, about 4 mm to about 10 mm, about 4 mm to about 20 mm, about 4 mm to about 30 mm, about 4 mm to about 40 mm, about 4 mm to about 50 mm, about 4 mm to about 75 mm, about 4 mm to about 100 mm, about 5 mm to about 7 mm, about 5 mm to about 10 mm, about 5 mm to about 20 mm, about 5 mm to about 30 mm, about 5 mm to about 40 mm, about 5 mm to about 50 mm, about 5 mm to about 75 mm, about 5 mm to about 100 mm, about 7 mm to about 10 mm, about 7 mm to about 20 mm, about 7 mm to about 30 mm, about 7 mm to about 40 mm, about 7 mm to about 50 mm, about 7 mm to about 75 mm, about 7 mm to about 100 mm, about 10 mm to about 20 mm, about 10 mm to about 30 mm, about 10 mm to about 40 mm, about 10 mm to about 50 mm, about 10 mm to about 75 mm, about 10 mm to about 100 mm, about 20 mm to about 30 mm, about 20 mm to about 40 mm, about 20 mm to about 50 mm, about 20 mm to about 75 mm, about 20 mm to about 100 mm, about 30 mm to about 40 mm, about 30 mm to about 50 mm, about 30 mm to about 75 mm, about 30 mm to about 100 mm, about 40 mm to about 50 mm, about 40 mm to about 75 mm, about 40 mm to about 100 mm, about 50 mm to about 75 mm, about 50 mm to about 100 mm, or about 75 mm to about 100 mm. In some aspects, the gap width is about 2 mm, about 3 mm, about 4 mm, about 5 mm, about 7 mm, about 10 mm, about 20 mm, about 30 mm, about 40 mm, about 50 mm, about 75 mm, or about 100 mm. In some aspects, the gap width is at least about 2 mm, about 3 mm, about 4 mm, about 5 mm, about 7 mm, about 10 mm, about 20 mm, about 30 mm, about 40 mm, about 50 mm, or about 75 mm. In some aspects, the gap width is at most about 3 mm, about 4 mm, about 5 mm, about 7 mm, about 10 mm, about 20 mm, about 30 mm, about 40 mm, about 50 mm, about 75 mm, or about 100 mm.

In some aspects, the open portion 601 can be described as having a height measured from the contact surface 701 to the interface 608 between the opened portion 601 and the elongated body 603.

In some aspects, the height of the opened portion is about 2 mm to about 100 mm. In some aspects, the height of the opened portion is about 2 mm to about 3 mm, about 2 mm to about 4 mm, about 2 mm to about 5 mm, about 2 mm to about 7 mm, about 2 mm to about 10 mm, about 2 mm to about 20 mm, about 2 mm to about 30 mm, about 2 mm to about 40 mm, about 2 mm to about 50 mm, about 2 mm to about 75 mm, about 2 mm to about 100 mm, about 3 mm to about 4 mm, about 3 mm to about 5 mm, about 3 mm to about 7 mm, about 3 mm to about 10 mm, about 3 mm to about 20 mm, about 3 mm to about 30 mm, about 3 mm to about 40 mm, about 3 mm to about 50 mm, about 3 mm to about 75 mm, about 3 mm to about 100 mm, about 4 mm to about 5 mm, about 4 mm to about 7 mm, about 4 mm to about 10 mm, about 4 mm to about 20 mm, about 4 mm to about 30 mm, about 4 mm to about 40 mm, about 4 mm to about 50 mm, about 4 mm to about 75 mm, about 4 mm to about 100 mm, about 5 mm to about 7 mm, about 5 mm to about 10 mm, about 5 mm to about 20 mm, about 5 mm to about 30 mm, about 5 mm to about 40 mm, about 5 mm to about 50 mm, about 5 mm to about 75 mm, about 5 mm to about 100 mm, about 7 mm to about 10 mm, about 7 mm to about 20 mm, about 7 mm to about 30 mm, about 7 mm to about 40 mm, about 7 mm to about 50 mm, about 7 mm to about 75 mm, about 7 mm to about 100 mm, about 10 mm to about 20 mm, about 10 mm to about 30 mm, about 10 mm to about 40 mm, about 10 mm to about 50 mm, about 10 mm to about 75 mm, about 10 mm to about 100 mm, about 20 mm to about 30 mm, about 20 mm to about 40 mm, about 20 mm to about 50 mm, about 20 mm to about 75 mm, about 20 mm to about 100 mm, about 30 mm to about 40 mm, about 30 mm to about 50 mm, about 30 mm to about 75 mm, about 30 mm to about 100 mm, about 40 mm to about 50 mm, about 40 mm to about 75 mm, about 40 mm to about 100 mm, about 50 mm to about 75 mm, about 50 mm to about 100 mm, or about 75 mm to about 100 mm. In some aspects, the height of the opened portion is about 2 mm, about 3 mm, about 4 mm, about 5 mm, about 7 mm, about 10 mm, about 20 mm, about 30 mm, about 40 mm, about 50 mm, about 75 mm, or about 100 mm. In some aspects, the height of the opened portion is at least about 2 mm, about 3 mm, about 4 mm, about 5 mm, about 7 mm, about 10 mm, about 20 mm, about 30 mm, about 40 mm, about 50 mm, or about 75 mm. In some aspects, the height of the opened portion is at most about 3 mm, about 4 mm, about 5 mm, about 7 mm, about 10 mm, about 20 mm, about 30 mm, about 40 mm, about 50 mm, about 75 mm, or about 100 mm.

In some aspects, the portion of the applicator tip which defines the open-shape corresponds to an arc of about 30° to about 300°. In some aspects, the portion of the applicator tip which defines the opening of the c-shape corresponds to an arc of about 30° to about 40°, about 30° to about 45°, about 30° to about 60°, about 30° to about 90°, about 30° to about 120°, about 30° to about 150°, about 30° to about 180 °, about 30° to about 210°, about 30° to about 240°, about 30° to about 270°, about 30° to about 300°, about 40° to about 45°, about 40° to about 60°, about 40° to about 90°, about 40° to about 120°, about 40° to about 150°, about 40° to about 180°, about 40° to about 210°, about 40° to about 240°, about 40° to about 270°, about 40° to about 300°, about 45° to about 60°, about 45° to about 90°, about 45° to about 120°, about 45° to about 150°, about 45° to about 180 °, about 45° to about 210°, about 45° to about 240°, about 45° to about 270°, about 45° to about 300°, about 60° to about 90°, about 60° to about 120°, about 60° to about 150°, about 60° to about 180°, about 60° to about 210°, about 60° to about 240°, about 60° to about 270°, about 60° to about 300°, about 90° to about 120°, about 90° to about 150°, about 90° to about 180 °, about 90° to about 210°, about 90° to about 240°, about 90° to about 270°, about 90° to about 300°, about 120° to about 150°, about 120° to about 180°, about 120° to about 210°, about 120° to about 240°, about 120° to about 270°, about 120° to about 300°, about 150° to about 180°, about 150° to about 210°, about 150° to about 240°, about 150° to about 270°, about 150° to about 300°, about 180° to about 210°, about 180° to about 240°, about 180 ° to about 270°, about 180° to about 300°, about 210° to about 240°, about 210° to about 270°, about 210° to about 300°, about 240° to about 270°, about 240° to about 300°, or about 270° to about 300°. In some aspects, the portion of the applicator tip which defines the opening of the c-shape corresponds to an arc of about 30°, about 40°, about 45°, about 60°, about 90°, about 120°, about 150°, about 180°, about 210°, about 240°, about 270°, or about 300°. In some aspects, the portion of the applicator tip which defines the opening of the c-shape corresponds to an arc of at least about 30°, about 40°, about 45°, about 60°, about 90°, about 120°, about 150°, about 180°, about 210°, about 240°, or about 270°. In some aspects, the portion of the applicator tip which defines the opening of the c-shape corresponds to an arc of at most about 40°, about 45°, about 60°, about 90°, about 120°, about 150°, about 180°, about 210°, about 240°, about 270°, or about 300°.

The opened portion can employ various opened cross-sectional contact surface shapes including but not limited to circular, elliptical, triangular, rectangular, pentagonal, hexagonal, heptagonal, octagonal, nonagonal, or decagonal.

In some aspects, the inclined applicator tips comprise an elongated body portion 603 having a length, width and height. In some aspects, the elongated body portion is coupled to the opened portion. In some aspects, the elongated body portion is coupled to the opened portion such that the longest dimension of the elongated body portion forms an angle of inclination (θ) with respect to the plane which is parallel to the c-shape of the opened portion.

In some aspects, the angle of inclination (θ) is about 30° to about 90°. In some aspects, the angle of inclination (θ) is about 30° to about 40°, about 30° to about 45°, about 30° to about 50°, about 30° to about 55°, about 30° to about 60°, about 30° to about 65°, about 30° to about 70°, about 30° to about 75°, about 30° to about 80°, about 30° to about 85°, about 30° to about 90°, about 40° to about 45°, about 40° to about 50°, about 40° to about 55°, about 40° to about 60°, about 40° to about 65°, about 40° to about 70°, about 40° to about 75°, about 40° to about 80°, about 40° to about 85°, about 40° to about 90°, about 45° to about 50°, about 45° to about 55°, about 45° to about 60°, about 45° to about 65°, about 45° to about 70°, about 45° to about 75°, about 45° to about 80°, about 45° to about 85°, about 45° to about 90°, about 50° to about 55°, about 50° to about 60°, about 50° to about 65°, about 50° to about 70°, about 50° to about 75°, about 50° to about 80°, about 50° to about 85°, about 50° to about 90°, about 55° to about 60°, about 55° to about 65°, about 55° to about 70°, about 55° to about 75°, about 55° to about 80°, about 55° to about 85°, about 55° to about 90°, about 60° to about 65°, about 60° to about 70°, about 60° to about 75°, about 60° to about 80°, about 60° to about 85°, about 60° to about 90°, about 65° to about 70°, about 65° to about 75°, about 65° to about 80°, about 65° to about 85°, about 65° to about 90°, about 70° to about 75°, about 70° to about 80°, about 70° to about 85°, about 70° to about 90°, about 75° to about 80°, about 75° to about 85°, about 75° to about 90°, about 80° to about 85°, about 80° to about 90°, or about 85° to about 90°. In some aspects, the angle of inclination (θ) is about 30°, about 40°, about 45°, about 50°, about 55°, about 60°, about 65°, about 70°, about 75°, about 80°, about 85°, or about 90°. In some aspects, the angle of inclination (θ) is at least about 30°, about 40°, about 45°, about 50°, about 55°, about 60°, about 65°, about 70°, about 75°, about 80°, or about 85°. In some aspects, the angle of inclination (θ) is at most about 40°, about 45°, about 50°, about 55°, about 60°, about 65°, about 70°, about 75°, about 80°, about 85°, or about 90°.

In some aspects, the longest dimension of the elongated body 603, which the length of the elongated body measured in parallel to the cooling axis of the cooling device when the cooling tip is attached is about 15 mm to about 500 mm. In some aspects, the longest dimension of the elongated body is about 15 mm to about 30 mm, about 15 mm to about 40 mm, about 15 mm to about 50 mm, about 15 mm to about 60 mm, about 15 mm to about 80 mm, about 15 mm to about 100 mm, about 15 mm to about 125 mm, about 15 mm to about 150 mm, about 15 mm to about 200 mm, about 15 mm to about 250 mm, about 15 mm to about 500 mm, about 30 mm to about 40 mm, about 30 mm to about 50 mm, about 30 mm to about 60 mm, about 30 mm to about 80 mm, about 30 mm to about 100 mm, about 30 mm to about 125 mm, about 30 mm to about 150 mm, about 30 mm to about 200 mm, about 30 mm to about 250 mm, about 30 mm to about 500 mm, about 40 mm to about 50 mm, about 40 mm to about 60 mm, about 40 mm to about 80 mm, about 40 mm to about 100 mm, about 40 mm to about 125 mm, about 40 mm to about 150 mm, about 40 mm to about 200 mm, about 40 mm to about 250 mm, about 40 mm to about 500 mm, about 50 mm to about 60 mm, about 50 mm to about 80 mm, about 50 mm to about 100 mm, about 50 mm to about 125 mm, about 50 mm to about 150 mm, about 50 mm to about 200 mm, about 50 mm to about 250 mm, about 50 mm to about 500 mm, about 60 mm to about 80 mm, about 60 mm to about 100 mm, about 60 mm to about 125 mm, about 60 mm to about 150 mm, about 60 mm to about 200 mm, about 60 mm to about 250 mm, about 60 mm to about 500 mm, about 80 mm to about 100 mm, about 80 mm to about 125 mm, about 80 mm to about 150 mm, about 80 mm to about 200 mm, about 80 mm to about 250 mm, about 80 mm to about 500 mm, about 100 mm to about 125 mm, about 100 mm to about 150 mm, about 100 mm to about 200 mm, about 100 mm to about 250 mm, about 100 mm to about 500 mm, about 125 mm to about 150 mm, about 125 mm to about 200 mm, about 125 mm to about 250 mm, about 125 mm to about 500 mm, about 150 mm to about 200 mm, about 150 mm to about 250 mm, about 150 mm to about 500 mm, about 200 mm to about 250 mm, about 200 mm to about 500 mm, or about 250 mm to about 500 mm. In some aspects, the longest dimension of the elongated body is about 15 mm, about 30 mm, about 40 mm, about 50 mm, about 60 mm, about 80 mm, about 100 mm, about 125 mm, about 150 mm, about 200 mm, about 250 mm, or about 500 mm. In some aspects, the longest dimension of the elongated body is at least about 15 mm, about 30 mm, about 40 mm, about 50 mm, about 60 mm, about 80 mm, about 100 mm, about 125 mm, about 150 mm, about 200 mm, or about 250 mm. In some aspects, the longest dimension of the elongated body is at most about 30 mm, about 40 mm, about 50 mm, about 60 mm, about 80 mm, about 100 mm, about 125 mm, about 150 mm, about 200 mm, about 250 mm, or about 500 mm.

In some aspects, the inclined applicator tip can be operably coupled to a cooling device by a mounting portion 605 mounted to the elongated portion 603, opposite to the contact surface. The mounting portion can comprise one or more coupling elements 607 designed to operably couple the applicator tip to the cooling device. The coupling elements can comprise posts, screws, clips, anchors, rivets, magnets, or other means of physically fastening the cooling tip to the device. The mounting portion can comprise a rotatable mount, which can be used to adjust the orientation of the contact surface with respect to the longitudinal cooling axis of the cooling device. In some aspects, the rotatable mount can be configured to allow a plurality of discreet mounting orientations, for example three mounting orientations can be achieved by rotating the coupling elements into each of three different positions. In some aspects, the rotatable mount can be configured to allow a continuum of mounting orientations, for example to allow free rotation about the longitudinal cooling axis of the cooling device. In some aspects, a rotatable mount configured to allow a continuum of mounting orientations comprises a locking mechanism to fix the orientation in a selected position.

In some aspects, the inclined applicator tip can be a disposable, single use applicator tip. In some aspects, the inclined applicator tip can be a reusable applicator tip which can be sterilized or recycled.

### Use of Inclined Applicator Tip

In some aspects, the inclined applicator tip provides improved access to the cooling area while using a cooling device. In some aspects, the improved access allows for easier combination of cooling treatment (e.g., cooling therapy, cooling anesthesia, analgesic cooling, etc.) with other types of treatment or therapy. In some aspects, the other types of treatment or therapy can be an injection, a laser therapy, a light-exposure therapy, radiotherapy, biopsy, surgery, or a combination thereof. In some aspects, use of the cooling therapy device in combination with the other type of therapy reduces pain associated with the other type of therapy. In some aspects, use of the cooling therapy device in combination with the other type of therapy increases the efficacy associated with the other type of therapy. In some aspects, co-administration of cooling therapy and one or more other types of therapy are applied simultaneously, or near simultaneously. In some aspects, the target area where the cooling therapy or co-administered therapies are to be applied is pre-marked, such as using a dye, a pen, tape, or other means of identifying a specific target site.

In some aspects, wherein the cooling device is a cryogenic cooling device, the angle of inclination is set to an optimum angle such that access is maximized (at larger angles), while loss of cryogen flow due to gravity is minimized (at angles which keep the cooling axis perpendicular to a horizontal target site, or parallel to a vertical target site). In some aspects, the optimum angle of inclination is no more than about 60 degrees for a vertical target site. In some aspects, the optimum angle of inclination depends on the orientation of the target site with respect to the gravitational force vector acting on the cryogen. In some aspects, the angle of inclination is chosen such that the angle between the longest dimension of a cryogen canister and the axis gravitational force vector is no more than about 60 degrees and the canister is oriented such that the outlet port is lower in elevation than the opposite end of the canister.

In some aspects, described herein are methods of cooling a target surface using a handheld cooling device equipped with an inclined applicator tip. In some aspects, the method can include providing an inclined applicator tip that is adapted to be operably coupled to the handheld cooling device. The method can comprise selecting an orientation of the opened portion with respect to a cooling axis of the handheld cooling device, wherein the orientation of the opened portion forms an angle of inclination (θ) with respect to the cooling axis. The method can comprise using the cooling device to cool a target surface by delivering the cooling medium to the target surface along the cooling axis.

In some aspects, selection is performed by mounting the applicator tip to the handheld cooling device in a position selected from three discreet rotational orientations. In some aspects, the selection is performed by selecting an orientation from a continuum of rotational orientations using a rotatable mount of the handheld cooling device.

In some aspects, the cooling device is a cryogenic cooling device wherein the cooling medium is a cryogen, which is selected from liquid helium, liquid nitrogen, liquid argon, liquid CO₂, or liquid O₂.

The method can include selecting an orientation of the cryogenic cooling device to minimize a reduction of cryogen flow into the cooling device caused by gravity. The method can further comprise applying a treatment to the target surface concurrently or near simultaneously with the cooling. The method can include treating a subject by applying cooling and an injection, a laser treatment, a surgery, or a drug application. The method can include treatment that is performed through the opened portion of the inclined applicator tip.

### EXAMPLES

### Example 1: Use of a Cryogenic Therapy Device with an Inclined Applicator Tip in Combination Therapy

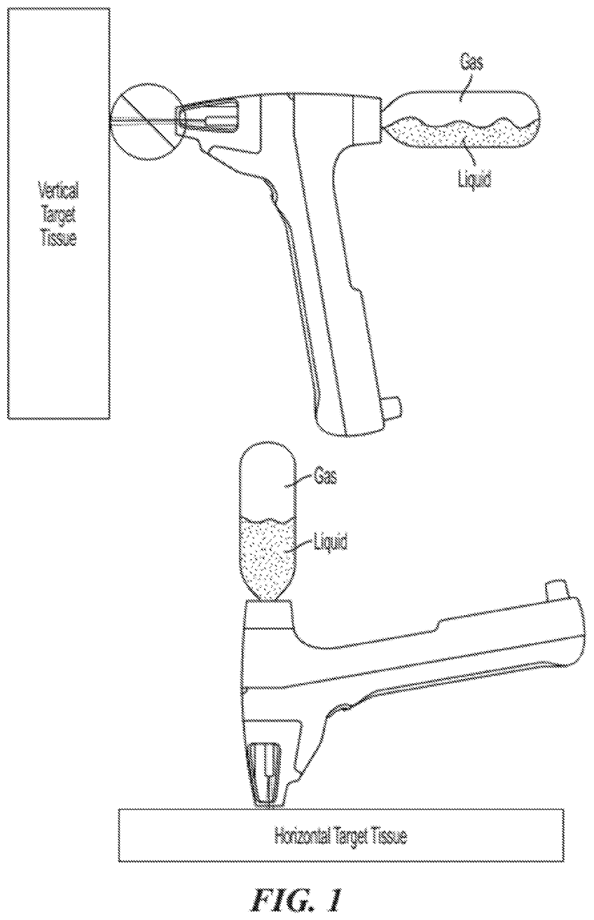

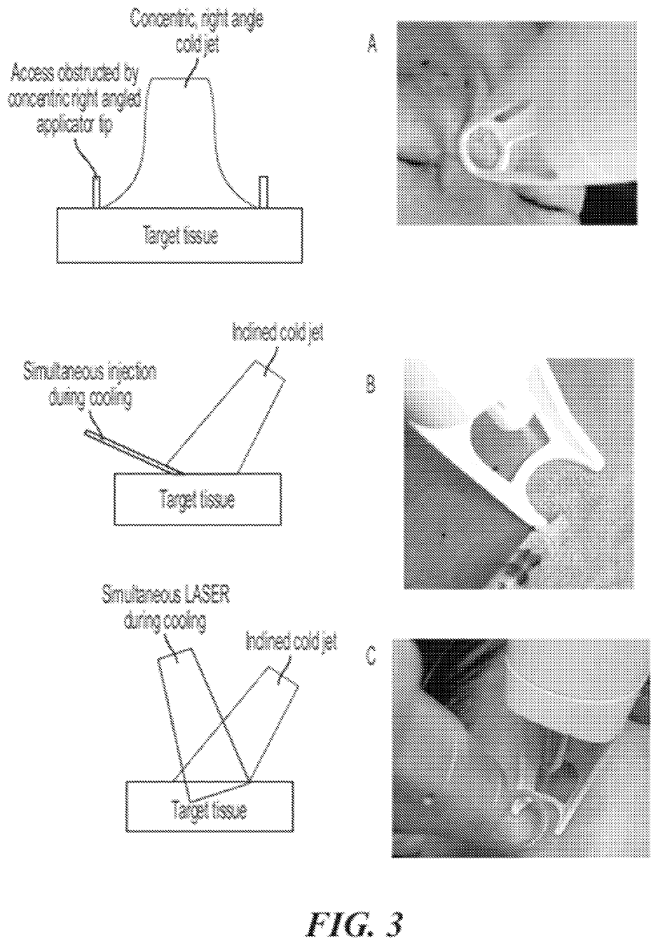

A medical professional wants to co-administer cooling therapy and an injection using a cryogenic cooling device. A cryogenic cooling device equipped with a non-inclined applicator tip and a cryogen canister is used to apply a cooling jet to a target site along a cooling axis, as seen in FIG. 1. The enclosed non-inclined applicator tip prevents liquid cryogen from entering the nozzle by gravity (by making the angle too steep as seen in FIG. 1, top panel) and blocks access to the target site for subsequent injections. In order to use the device, the target site must be oriented close to horizontal (as seen in FIG. 1, lower panel and FIG. 3A), and the cryogenic cooling device must be removed prior to administration of an injection or other subsequent therapy.

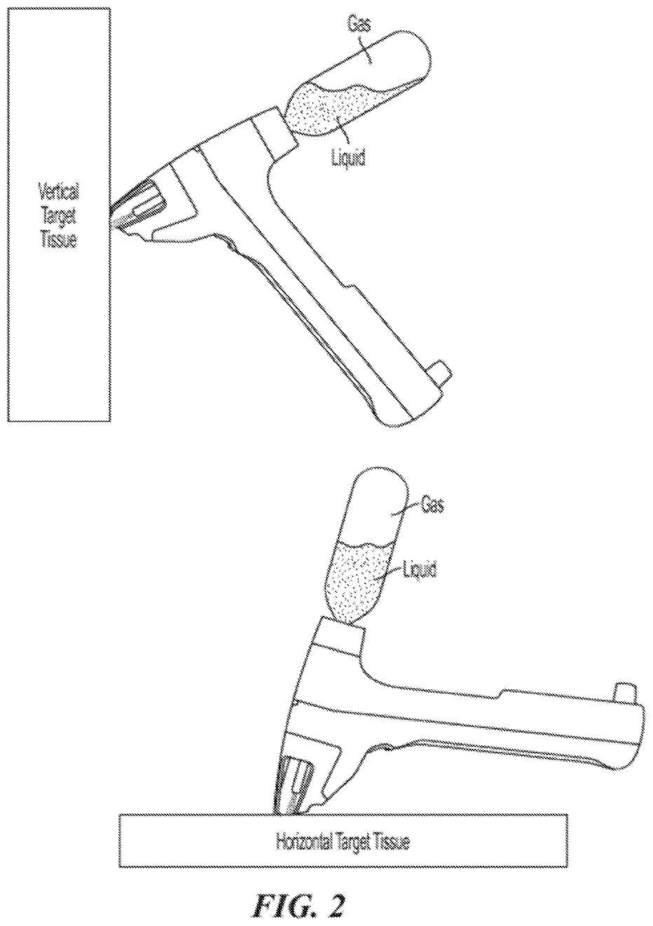

When the medical professional uses a cryogenic cooling device equipped with an inclined applicator tip, as see in FIG. 2, they can rotate the angle of inclination about the cooling axis of the device such that the cryogenic cooling device will operate on target tissues at any angle. The angle of inclination of the applicator tip is chosen to be as large as possible to maximize access (by placing the cooling farther out of the way of the target site) such that liquid cryogen is not prevented from entering the nozzle by gravity.

The temperature of a cryogen is controlled forming a cold jet which is applied using an inclined applicator tip to produce a temperature cold enough to immediately anesthetize a target area on a subject, while allowing access to the target site. Reduction of the temperature mitigates pain in the target site during hypodermic injection FIG. 3B and/or laser procedures FIG. 3C, which are carried out while the cold jet is present, or a few seconds after, for example within 3to 5 seconds as illustrated in FIG. 3. The inclined applicator is designed to set a constant distance between the cryogen nozzle outlet to promote precision temperature control. The inclined applicator tip has an opened contact surface mounted at an angle to allow maximum visibility and accessibility to the target area while cooling is taking place and/or shortly thereafter.

Accordingly, different angle of inclinations are chosen depending on whether the target area is vertical (such as target skin for injecting a vaccination into an arm of a standing person) or horizontal. The angle of inclination of a fixed inclined applicator tip can be adjusted with respect to a target site by rotating a rotatable mount on the device about the cooling axis in order to achieve an appropriate angle for the specific target site. FIG. 3B and FIG. 3C demonstrate simultaneous injection and simultaneous laser therapy while applying a cryotherapy device to reduce pain at the injection or laser application site. The open shaped ring of the inclined applicator tip maximizes the visibility and accessibility, particularly compared to concentric closed ring applicator tips, such as those shown in FIG. 3A, which place the device perpendicular to the target site.

### Example 2: An Opened Inclined Applicator Tip Designed to be Operably Coupled to a Cryotherapy Device

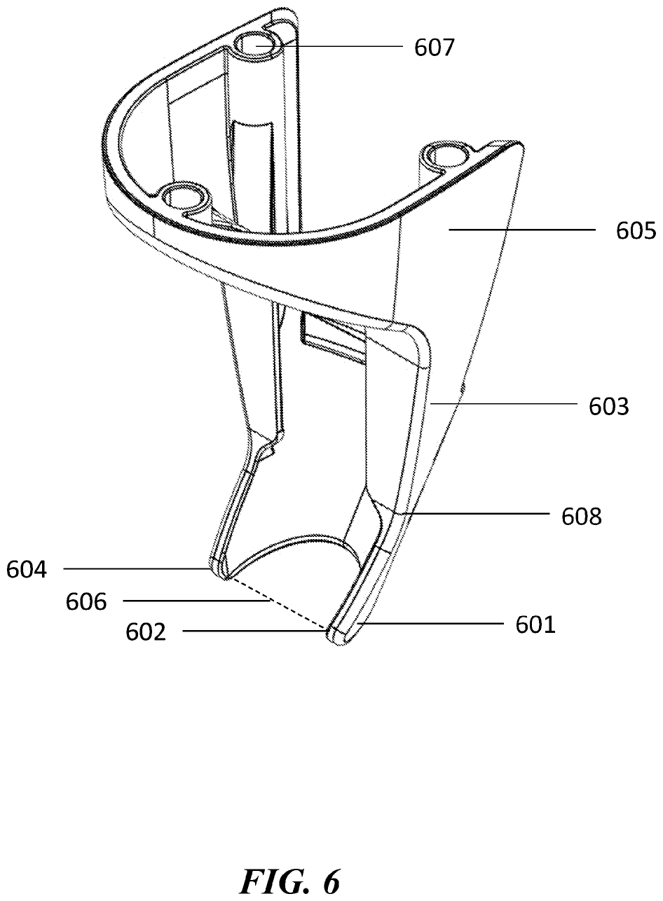

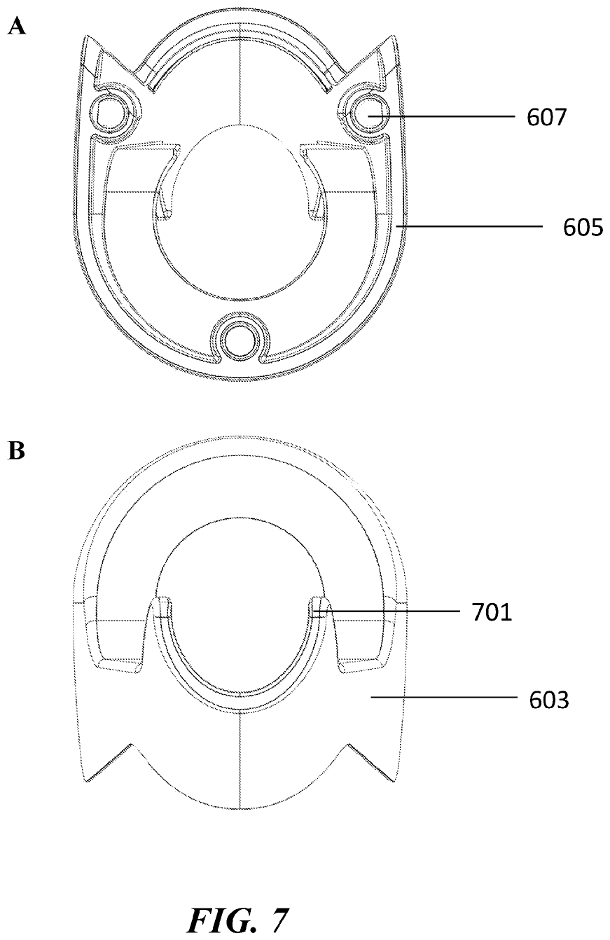

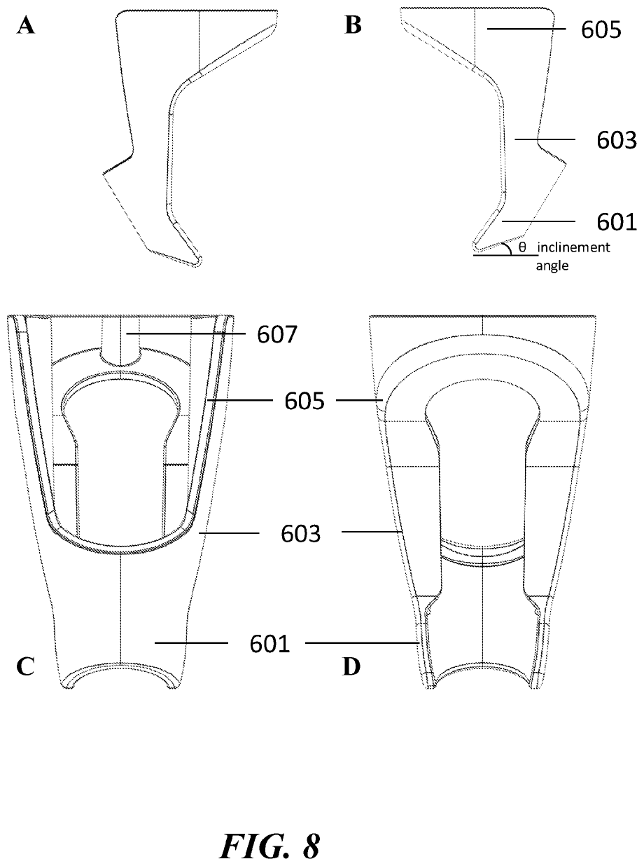

An inclined applicator tip was designed to couple with a cryocooling device to allow access to the target site by providing an angle of inclination and an opening for access, as shown in FIGS. 6-8. The example applicator tip has an opened contact portion 601, mounted to an elongated body portion 603 at an angle of inclination (θ), opposite to a mounting portion 605, which contains coupling elements 607 adapted to operably couple the example inclined applicator tip to an example cryogenic cooling device.

While preferred aspects of the present invention have been shown and described herein, it will be obvious to those skilled in the art that such aspects are provided by way of example only. It is not intended that the invention be limited by the specific examples provided within the specification. While the invention has been described with reference to the aforementioned specification, the descriptions and illustrations of the aspects herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the aspects of the invention described herein can be employed in practicing the invention. It is therefore contemplated that the invention shall also cover any such alternatives, modifications, variations, or equivalents. It is intended that the following claims define the scope of the invention and that methods and structures within the scope of these claims and their equivalents covered thereby.

1. An inclined applicator tip adapted to be operably coupled to a handheld cooling device, the inclined applicator tip comprising:
an opened portion, at least a portion of an edge of the opened portion comprising a c-shape opening;
an elongated body portion coupled to the opened portion-, a longest dimension of the elongated body portion forming an angle of inclination angle of inclination (θ) with respect to a plane which is parallel to the c-shape opening, the elongated body portion being substantially parallel to a cooling axis of the handheld cooling device when operably coupled; and
a mounting portion adapted to be operably coupled to the cooling device, the mounting portion and the opened portion being coupled to the elongated body portion at opposite ends of the longest dimension of the elongated body portion.

2. The inclined applicator tip of claim 1, wherein the handheld cooling device comprises a cryogenic cooling device.

3. The inclined applicator tip of claim 1, wherein the opened portion comprises a circular, an elliptical, or a polygonal shape.

4. The inclined applicator tip of claim 1, wherein the opened portion comprises a circular shape, and wherein a portion of the circular shape which forms the c-shape opening comprises a 90° arc.

5. The inclined applicator tip of claim 1, wherein the opened portion comprises a circular shape, and wherein a portion of the circular shape which forms the c-shape opening comprises a 180° arc.

6. The inclined applicator tip of claim 1, wherein the opened portion comprises a circular shape, and wherein a portion of the circular shape which forms the c-shape opening comprises a 270° arc.

7. The inclined applicator tip of claim 1, wherein the angle of inclination (θ) is about 60°.

8. The inclined applicator tip of claim 1, wherein the angle of inclination (θ) is about 70°.

9. The inclined applicator tip of claim 1, wherein the angle of inclination (θ) is about 80°.

10. The inclined applicator tip of claim 1, wherein the angle of inclination (θ) is about 90°.

11. The inclined applicator tip of claim 1, wherein the opened portion comprises an inner radius of at least 5 mm, 10 mm, 15 mm, 20 mm, 30 mm, or 50 mm.

12. The inclined applicator tip of claim 1, wherein the longest dimension of the elongated body portion is no more than about 250 mm.

13. The inclined applicator tip of claim 1, wherein the longest dimension of the elongated body portion is at least about 15 mm.

14. The inclined applicator tip of claim 1, wherein the angle of inclination (θ) is about 60°, and wherein the opened portion comprises an inner radius of at least 5 mm.

15. The inclined applicator tip of claim 1, wherein the mounting portion comprises a magnetic coupling element adapted to operably couple to the cooling device.

16. The inclined applicator tip of claim 15, wherein the magnetic coupling element comprises one or more permanent magnets.

17. The inclined applicator tip of claim 15, wherein the magnetic coupling element comprises a ferromagnetic material, and wherein the cooling device comprises one or more magnets configured to attract the ferromagnetic material in response to the inclined applicator tip being coupled to the device.

18. The inclined applicator tip of claim 1, wherein a minimum clearance distance is at least 5 mm, 10 mm, 15 mm, 20 mm, 30 mm, or 50 mm.

19. The inclined applicator tip of claim 1, wherein a minimum gap distance is about 5 mm, 7 mm, 10 mm, 15 mm, or 20 mm.

20. The inclined applicator tip of claim 1, wherein a height of the opened portion is at least 5 mm, 10 mm, 15 mm, 20 mm, 30 mm, or 50 mm.

21. A method of cooling a target surface using a handheld cooling device, the method comprising:
providing an inclined applicator tip adapted to be operably coupled to the handheld cooling device, wherein the inclined applicator tip comprises an opened portion, an elongated body portion coupled to the opened portion, and a mounting portion adapted to be operably coupled to the handheld cooling device;
selecting an orientation of the opened portion with respect to a cooling axis of the handheld cooling device, wherein the orientation of the opened portion forms an angle of inclination (θ) with respect to the cooling axis; and
cooling the target surface by delivering the cooling medium to the target surface along the cooling axis.

22. The method of claim 21, wherein the selecting is performed by mounting the applicator tip to the handheld cooling device in a position selected from three discreet rotational orientations.

23. The method of claim 21, wherein the selecting is performed by selecting an orientation from a continuum of rotational orientations using a rotatable mount of the handheld cooling device.

24. The method of claim 21, wherein the cooling medium comprises a cryogen.

25. The method of claim 24, wherein the angle of inclination and the user selected orientation are designed to minimize a reduction of cryogen flow into the cooling device caused by gravity.

26. The method of claim 21, further comprising applying a treatment to the target surface concurrently or near simultaneously with the cooling.

27. The method of claim 26, wherein the treatment comprises an injection, a laser treatment, a surgery, or a drug application.

28. The method of claim 26, wherein the treatment is performed through the opened portion of the inclined applicator tip.
